(19) United States
(12) Patent Application Publication
(10) Pub. No.: US 2005/0027134 A1
(43) Pub. Date: Feb. 3, 2005

(54) Support for a catalyst for direct oxidation of propylene to propylene oxide, method of making and method of using catalyst

(75) Inventors: Patricia A. Hooks, Houston, TX (US); David L. Sullivan, Houston, TX (US); Michael D. Mier, Waller, TX (US); Jaap W. van Hal, Fresno, TX (US); Xiankuan Zhang, Houston, TX (US)

Correspondence Address: Jim Wheelington;SABIC Americas, Inc., SABIC Technology Center, 1600 Industrial Blvd., Sugar Land, TX, 77478, US

(73) Assignee: Saudi Basic Industries Corporation

(21) Appl. No.: 10/902,627

(22) Filed: Jul. 29, 2004

Related U.S. Application Data
(60) Provisional application No. 60/490,693, filed on Jul. 29, 2003.

(51) Int. Cl. B01J 21/18; C07D 301/03

(52) U.S. Cl. 549/536; 502/174

(57) ABSTRACT

This invention is for a support for a silver-containing catalyst which can be used in a method of making an alkene oxide from an alkene and an oxygen-containing gas by oxidation of the alkene to the corresponding epoxide, such as propylene to propylene oxide. The support is an alkaline earth metal carbonate, such as calcium carbonate, and the shape of the support is not regular rhombohedral, cubic nor a blend of regular rhombohedral and cubic. Preferably, the shape of the support is scalenohedral, irregular rhombohedral, acicular or prismatic. The catalyst may contain optional promoters, such as potassium, chlorine, molybdenum, rhenium, tungsten, gold, thallium, yttrium, niobium, indium, barium, cobalt or cerium, and the support may contain an additional support material, such as an alkaline earth oxide.

### BACKGROUND OF THE INVENTION

1. Field of the Invention

This invention relates to a support for a silver-containing catalyst, a method of preparing such catalyst and a method of using such catalyst in a process of making an alkene oxide from an alkene and an oxygen-containing gas by oxidation of the alkene to the corresponding epoxide.

2. Description of the Prior Art

Producing an alkene oxide or epoxide, particularly propylene oxide, by direct oxidation of an alkene in the presence of a silver-containing catalyst is well known. The reaction ideally proceeds as follows:
2 CH₃CH═CH₂+O₂→2 CH₃CHOCH₂
but CO₂ and H₂O can be produced as well as some other minor byproducts.

Typically, the catalyst contains silver on a support. The catalyst may also contain small amounts of alkali metals, such as potassium, sodium, rubidium or cesium, and other metals, such as gold, tungsten, rhenium, molybdenum, fluorine, thallium, yttrium, barium, cerium, cobalt, indium or niobium, and halides, such as chlorine, as promoters to improve selectivity, activity, conversion, stability or yield.

Several supports for silver-containing catalysts used for propylene epoxidation are known. Canadian Patent no. 1282772 contains a discussion of many of the variables affecting catalyst performance, including the support. Material, physical and chemical properties, purity, phases and morphology are factors which are considered for the support. An alkaline earth metal carbonate in granular or crystalline form is disclosed as the preferred support.

U.S. Pat. No. 6,083,870 discloses vapor phase epoxidation of propylene to propylene oxide with a silver catalyst supported on certain alkaline earth metal compounds such as calcium titanate, barium titanate, magnesium titanate, tribasic calcium phosphate, calcium molybdate, calcium fluoride, magnesium aluminate and strontium titanate. Other materials, such as monobasic calcium phosphate, dibasic calcium phosphate, hydroxyapatite, tricalcium phosphate, were shown as examples of undesirable supports.

U.S. Pat. No. 5,703,254 discloses vapor phase oxidation of propylene to propylene oxide with a catalyst containing silver, gold and a potassium promoter supported on a carbonate of alkaline earth metal ion, such as calcium, strontium, magnesium or barium with calcium being most preferred. A granular form of the carbonate support is preferred.

U.S. Pat. No. 5,770,746 discloses a process for direct oxidation of propylene to propylene oxide in the vapor phase with a silver catalyst supported on an inert refractory solid such as alumina, silicon carbide, silica, zirconia, titania and an alkaline earth metal carbonate with calcium carbonate being most preferred.

U.S. Pat. No. 5,780,657 discloses a process for direct oxidation of propylene to propylene oxide in the vapor phase with a silver catalyst supported on alkaline earth metal carbonate or alkaline earth metal titanates with calcium carbonate being preferred.

U.S. Pat. No. 6,399,794 discloses an olefin epoxidation with a catalyst of a noble metal, such as gold, silver, platinum, palladium, iridium, ruthenium or osmium, and titanium zeolite, such as titanium silicate, in the presence of a modifier of calcium carbonate and carbon dioxide or ammonium bicarbonate. The olefin, oxygen and hydrogen are reacted in the presence of the modifier and the catalyst. If calcium carbonate is used as the modifier, carbon dioxide must be present and calcium carbonate is preferably present in the range of from about 50 ppm to about 10,000 ppm. The epoxidation process can be in the liquid phase, the gas phase or in the supercritical phase.

The development of novel supports which provide improved performance in the epoxidation process as compared with known materials would be advantageous. Selection of such materials is not precise. Not all support material perform equivalently as supports for silver catalysts in vapor phase epoxidation of propylene. Indeed, not even preferred support material perform equivalently in such a process.

### SUMMARY OF THE INVENTION

The invention provides a catalyst for producing propylene oxide from propylene and oxygen, a method of making the catalyst and a method of using the catalyst. The catalyst comprises

a) a support of alkaline earth carbonate

b) a catalytically effective amount of silver

c) optionally, promoters selected from the group consisting of potassium, chlorine, molybdenum, rhenium, tungsten, gold, thallium, yttrium, niobium, indium, barium, cobalt or cerium. The support is an inorganic carbonate of the general formula ACO₃ where A is calcium, strontium, magnesium or barium with calcium being the most preferred. The shape of the support is not regular rhombohedral or cubic or a blend containing regular rhombohedral or cubic. Preferably, the shape of the support is scalenohedral, irregular rhombohedral, acicular or prismatic.

The alkaline earth carbonate support may be contacted with a solution, slurry, paste or gel containing a silver compound and, optionally, compounds of the promoters and the catalyst is then dried and calcined. Alternatively, the compounds of the promoters can be contacted with the calcined catalyst in a solution, slurry, paste or gel after which the catalyst is then dried. The catalyst may be formed into shapes suitable for a reactor in which to selectively convert propylene to propylene oxide.

The catalyst is brought into contact with propylene and oxygen under reaction conditions to selectively convert propylene to propylene oxide.

### BRIEF DESCRIPTION OF THE DRAWINGS

A more complete appreciation of the invention and many of the attendant advantages thereof will be readily understood by reference to the following detailed description when considered in connection with the accompanying drawings:

FIG. 1 is a scanning electron microscope (SEM) photograph of calcium carbonate having a scalenohedral shape (Tradename: Mallinckrodt 4052).

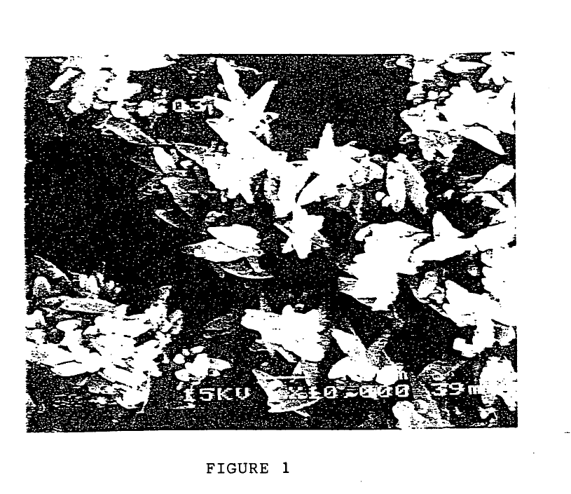

FIG. 2 is a scanning electron microscope (SEM) photograph of calcium carbonate having a regular rhombohedral shape (Tradename: Strem #93-2011).

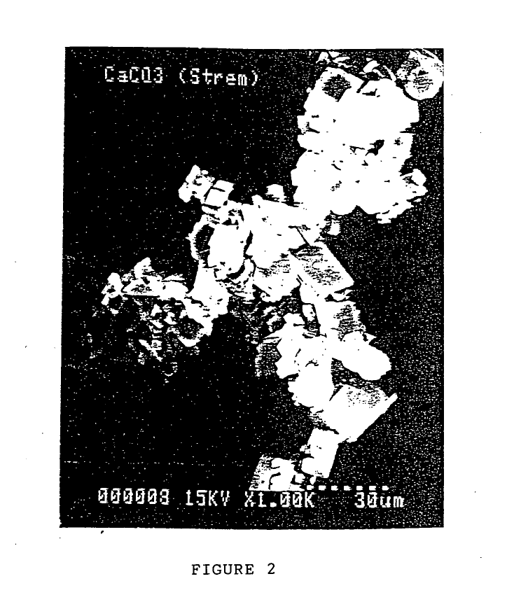

FIG. 3 is a scanning electron microscope (SEM) photograph of calcium having a regular rhombohedral shape (Tradename: Alfa #36337).

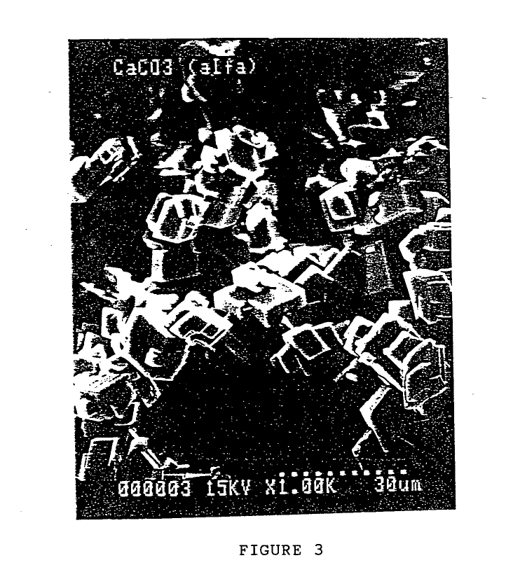

### DETAILED DESCRIPTION OF THE INVENTION

The present invention is for a silver-containing catalyst, a method of preparing such a catalyst and a method of using such a catalyst in a process of making an alkene oxide from an alkene and an oxygen-containing gas by oxidation of the alkene to the corresponding epoxide. The catalytically active silver is supported on an alkaline earth carbonate.

In this Specification, including the Claims, certain terms are used with the following meaning and definitions. The term “alkaline earth metal” refers to elements of Group 2 of the Periodic Table, i.e., beryllium, magnesium, calcium, strontium, barium and radium.

The term “support” is a carrier on which the catalytically active components of a heterogeneous catalyst are deposited.

The term “promoter” is a component of a catalyst that provides improvement in one or more of the properties of the catalyst, e.g., selectivity, activity, conversion, stability and yield as compared to a catalyst not containing the promoter.

The alkaline earth carbonate support is of the general formula ACO₃ where A is beryllium, calcium, strontium, magnesium, barium or radium with calcium being the most preferred. The shape of the support is not regular rhombohedral, cubic or a blend containing regular rhombohedral or cubic. Preferably, the shape of the support is scalenohedral, irregular rhombohedral, acicular or prismatic. Surface area is preferably from about 1 m²/g to about 30 m²/g. “Scalenohedral” refers to a solid having three unequal sides on its face. “Rhombohedral” refers to a solid having four sides on its face, regular with the four sides being equal and irregular with at least one side not being equal to the others. “Prismatic” refers to a solid having three sides on its face (like a prism), regular with the three sides being equal and irregular with at least one side not being equal to the others. “Cubic” refers to a solid in the shape of a cube. “Acicular” refers to a solid having a needle-like shape.

Calcium carbonate is a white crystalline solid that is one of the most common natural substances, forming chalk, limestone, and marble, and occurs in animal shells and bones. It occurs in natural crystal forms of calcite, which is trigonal, and aragonite, which is orthorhombic, and vaterite, an unstable form which will transform into calcite or aragonite over time. These crystal forms are specific mineral phases (morphologies) related to the distinct arrangement of the calcium, carbon and oxygen atoms in the crystal structure. These crystal structures can be formed into different shapes and symmetries, such as rhombohedral, scalenohedral, prismatic and spherical for calcite and acicular for aragonite.

Naturally-occurring calcium carbonate commonly contains some impurities such as iron, magnesium, strontium, barium, lead, and occasionally, sodium, potassium and sulfur. The impurities may exist separately or together in any combination. These elements or others may be added as modifiers. The modifiers or impurities may be present up to about 5%.

One method to synthesize calcium carbonate is to mix quicklime (CaO) with water to form a slurry and add carbon dioxide gas. The resulting reaction produces a very fine precipitated calcium carbonate. Another synthesis method for calcium carbonate is to react sodium carbonate (and, optionally, magnesium carbonate) with calcium chloride.

An additional support material may be included with the alkaline earth carbonate, such as an alkaline earth oxide of the general formula BO where B is beryllium, calcium, strontium, magnesium, barium or radium with calcium being the most preferred. A and B may be the same or different.

In general, the catalyst is prepared by adding a silver compound to a liquid to form a solution, slurry, paste or gel, contacting the solution, slurry, paste or gel with support particles, removing the liquid, drying the catalyst particles and reducing the silver compound to elemental silver. The silver compound can be an oxide, a salt or carboxylate. Examples of the silver compound are silver oxide, silver nitrate, silver carbonate, silver acetate, silver propionate, silver butyrate, silver oxalate, silver malonate, silver malate, silver maleate, silver lactate, silver citrate, and silver phthalate. The silver concentration in the finished catalyst is at least a catalytically effective amount, preferably from about 2 percent to 80 percent by weight, more preferably from about 10 percent to 70 percent by weight, most preferably from about 30 percent to 70 percent by weight and specifically about 54% by weight.

Optional promoters such as compounds of alkali metals, other metals or halides may be added to the solution, slurry, paste or gel or may be added to the solid catalyst after reduction. Examples of alkali metal promoters are potassium, sodium, rubidium or cesium, which can be added as salts, preferably carbonates, nitrates or nitrites, most preferably potassium nitrate. Examples of other metal promoters are gold, tungsten, rhenium, molybdenum, thallium, yttrium, barium, cerium, cobalt, indium and niobium may also be optionally added as promoters. These metals may be added as compounds such as oxides, acids, carbonates, sulfates, halides, oxyhalides, hydroxyhalides, hydroxides and sulfides. Examples of halide promoters are fluorine or chlorine which can be added as compounds such as silver fluoride or silver chloride. The halide promoter may be omitted if the feedstream contains a halide compound.

Any of these promoters (halide, alkali metal or other metals) may be added with the silver compound, to the solid catalyst after drying or to the calcined catalyst. The promoters are present in the catalyst in the amount of from about 0.1 to 5% by weight. The alkali metal is preferably present in the amount of from about 1 to 5% by weight, more preferably about 3% by weight. The halide is preferably present in the amount from about 0.01 to 1.0% by weight, more preferably 0.05 to 0.5% by weight. The other metals are preferably present in the amount from about 0.1 to 5.0% by weight, preferably from about 0.1 to 2% by weight.

Adding acid assists in the dissolution of the components to form a solution. Examples of the acid are organic acids, such as oxalic acid, propionic acid, malonic acid, citric acid, glycolic acid or mixtures thereof.

Liquid is removed from the solution, slurry, paste or gel to form solid particles of catalyst. The liquid may be removed by filtration, evaporation or spray drying.

The solid particles of catalyst may be dried in air or an inert gas at room or elevated temperature. Drying time may be from one hour to twenty-four hours, preferably one to four hours. Drying temperature may be from 110° C. to 250° C., preferably about 250° C. Drying is most preferably for four hours at 250° C.

The solid particle of catalyst may be sieved or formed by techniques known in the art to obtain desired sized and shape.

The catalyst must be calcined to further dry the catalyst and support, react the components and remove volatile compounds to have an effective catalyst for epoxidation of an alkene to an alkene oxide. Calcination should be at a temperature of from about 100° C. to about 500° C. for a time of from about one hour to about four hours. Calcination may be in one stage or multiple stages. For example, calcination may be at a temperature of about 250° C. for six hours or at a temperature of 110° C. for one hours and then increasing the temperature by 5° C./min to a temperature of 300° C. for additional calcination for four hours. Calcination is preferably at a temperature of 300° C. for a time of four hours. A reducing agent, such as hydrogen, may also be used during calcination. Without the present invention and its claims being limited by theory, it believed that exposing the catalyst to these elevated temperatures reduces the silver to its elemental form but that while other components (alkali earth metal carbonate, alkali metals, other metals or halides) may react during calcination they are not reduced to their elemental form.

The catalyst is brought into contact with propylene and oxygen under reaction conditions to selectively convert propylene to propylene oxide. Typical conditions for the epoxidation reaction are temperatures from about 180° C. to 350° C., preferably 200° C. to 300° C., and pressures from about 1 atmosphere to about 30 atmospheres, preferably about 1 atmosphere to about 5 atmospheres; however, commercial conditions may be from about 10 atmospheres to about 20 atmospheres. Propylene is present in the amount from about 2 to about 50% by volume, preferably 10 to 30% by volume, more preferably from about 10% to about 20% by volume. Oxygen is present in amount from about 2 to about 50% by volume, preferably 10 to 25%, more preferably about 15% by volume. The feedstream may optionally contain carbon dioxide, a gaseous nitrogen oxide species and a halide compound, preferably an organic halide. Carbon dioxide may be present in the amount from about 1 to about 50% by volume, preferably from about 5 to about 50% by volume, most preferably about 10% by volume. Examples of the gaseous nitrogen oxide species are nitrogen dioxide (NO₂), nitric oxide (NO), nitrogen peroxide (N₂O₄) and nitrogen trioxide (N₂O₃). Preferably, the gaseous nitrogen oxide species is nitric oxide. The gaseous nitrogen oxide species may be present in the feedstream in the amount of from 1 to 2000 ppm, preferably 20 to 500 ppm, more preferably about 50 to about 200 ppm. Examples of the organic halide are alkyl halides, such as ethylene dichloride, ethyl chloride, vinyl chloride, methyl chloride and methylene chloride. Preferably, the organic halide is ethyl chloride, ethylene dichloride or vinyl chloride, more preferably ethyl chloride. The organic halide is present in the feedstream in the amount of from about 1 to 2000 ppm, preferably 20 to 500 ppm, more preferably about 50 to 500 ppm.

The invention having been generally described, the following examples are given as particular embodiments of the invention to demonstrate the practice and advantages thereof. The reaction temperature varied between 220 and 260° C. to achieve about 10% propylene conversion. It is understood that the examples are given by way of illustration and are not intended to limit the specification or the claims in any manner.

CaCO₃ in a Scalenohedral Shape

### EXAMPLE 1

### Solution Preparation

Ethylenediamine (11.9 g) was added to a beaker (100 ml) with a stir bar and a temperature probe. Deionized water (14.0 g) was slowly added to the beaker while keeping the temperature at less than 50° C. Silver chloride (0.090 g) was added to the solution and stirred till completely dissolved. Oxalic acid (17.8 g) was slowly added to the solution while keeping the temperature at less than 40° C. Silver oxide (17.8 g) was slowly added to the solution while keeping the temperature at less than 50° C., and followed by adding ethanolamine (3.61 g). Calcium carbonate (11.6 g, Tradename: Mallinckrodt #4052) having a scalenohedral shape of 5.6 m²/g surface area was added to a ball-mill jar with 5 mixing stones. The silver-containing solution was poured from the beaker to the jar, and mixed well with the CaCO₃. A gel type of the mixture was formed after 3-8 minutes of mixing. It was aged for 1 hour prior to calcination that was carried out in a muffle furnace in air by drying at 110° C. for 1 hour and 130° C. for 1 hour, and then calcining at 300° C. for 3 hours. Potassium nitrate was added after calcination by dissolving KNO₃ (2.48 g) in 40 ml of deionized water in a round flask to which the powder of the catalyst precursor was then added. The mixture was dried in a rotary evaporator at 70° C. under vacuum for about 30 minutes and further dried in a muffle furnace at 250° C. for 4 hours. The catalyst was crushed, pressed, and sieved to 30-50 mesh prior to the use in epoxidation. The nominal composition of the catalyst was 54 wt. % Ag, 3 wt. % K, and 0.07 wt. % Cl on CaCO₃.

The freshly prepared catalyst was evaluated in a microreactor for propylene epoxidation using a feedstream containing 10 vol. % propylene, 11% vol. % oxygen, 10 vol. % carbon dioxide, 50 ppm ethylene chloride, 50 ppm nitric oxide, and balance methane at a total GHSV of 1200 h⁻¹ and a total pressure of 40 psig. After 20 hours on stream, propylene oxide selectivity was 61% and propylene conversion 10% at 239° C.

### EXAMPLE 2

### Slurry Preparation

Deionized water (40 ml) was added to a beaker (100 ml) with a stir bar and a temperature probe. Potassium nitrate (2.40 g) was added to the beaker and stirred till completely dissolved. Silver oxide (17.8) was added to the beaker and stirred for 10 minutes. 3.6 ml of ethylenediamine was added to the beaker, heated to 50° C., and stirred at 50° C. for 10 minutes. Separately, calcium carbonate (11.7 g, Tradename: Mallinckrodt #4052) having a scalenohedral shape was added to a ball-mill jar followed by pouring the slurry from the beaker. The slurry and calcium carbonate were mixed well using a Teflon rod, and allowed to sit for 1 hour before calcination as described in Example 1. The nominal composition of the catalyst is 54 wt. % Ag, and 3 wt. % K on CaCO₃.

The catalyst evaluation was similar to Example 1, except 15.5% O₂, and 200 ppm ethylchloride were used. After 95 hours on stream, propylene oxide selectivity was 60% and propylene conversion 10% at 238° C.

### EXAMPLE 3

A similar experiment as described in Example 2 was performed, but a calcium carbonate having a surface area of 7.5 m²/g (Tradename: Specialty Minerals Vicality Light) was used. Ethanolamine (3.6 ml) was used in replace of ethylenediamine. Potassium nitrate (2.24 g) was added after calcination as described in Example 1. After about 18 hours on stream, PO selectivity was 56% and propylene conversion 10% at 234° C.

### EXAMPLE 4

A similar experiment as described in Example 3 was performed, but a calcium carbonate having a surface area of 23.1 m²/g (Tradename: Specialty Minerals Albacar PO) was used. After 18 hours on stream, PO selectivity was 57% and propylene conversion 10% at 234° C.

CaCO₃ in a Irregular Rhombohedral Shape

### EXAMPLE 5

A similar experiment as described in Example 3 was performed, but a calcium carbonate having irregular rhombohedral shape (Tradename: Specialty Minerals Multifex MM) was used. After 18 hours on stream, PO selectivity was 57% and propylene conversion 10% at 237° C.

CaCO₃ in a Acicular Shape

### EXAMPLE 6

A similar experiment as described in Example 3 was performed, but a calcium carbonate having an acicular shape (Tradename: Specialty Minerals Opacarb A40) was used. After 20 hours on stream, PO selectivity was 55% and propylene conversion 11% at 244° C.

CaCO₃ in a Prismatic Shape

### EXAMPLE 7

A similar experiment as described in Example 3 was performed, but a calcium carbonate having a prismatic shape (Tradename: Specialty Minerals Albafil) was used. After 17 hours on stream, PO selectivity was 54% and propylene conversion 10% at 250° C.

CaCO₃ in a Regular Rhombohedral Shape

### COMPARATIVE EXAMPLE 1

A similar experiment as described in Example 1 was performed, but a calcium carbonate having a regular rhombohedral shape of 1.71 m²/g surface area (Tradename: Strem #93-2011) was used. After 78 hours on stream, PO selectivity was 53% and propylene conversion 3.6% at 250° C.

### COMPARATIVE EXAMPLE 2

A similar experiment as described in Example 1 was performed, but a calcium carbonate having a regular rhombohedral shape of 1.95 m²/g surface area (Tradename: Alfa #36337) was used. After 40 hours on stream, PO selectivity was 54% and propylene conversion 4.0% at 250° C.

CaCO₃ in a Cubic Shape

### COMPARATIVE EXAMPLE 3

A similar experiment as described in Example 3 was performed, but a calcium carbonate having a cubic shape of 0.32 m²/g surface area (Tradename: Mallinckrodt #4071) was used. After 20 hours on stream, PO selectivity was 30% and propylene conversion 2.9% at 260° C. COMPARATIVE EXAMPLE 4

A similar experiment as described in Example 1 was performed, but a calcium carbonate having a cubic shape of 1.48 m²/g surface area (Tradename: Franklin Calcite) was used. After 40 hours on stream, PO selectivity was 30% and propylene conversion 0.7% at 251° C.

Obviously, numerous modifications and variations of the present invention are possible in light of the above teachings. It is therefore to be understood that within the scope of the appended claims, the invention may be practiced otherwise than as specifically described herein.

1. A catalyst for oxidation of propylene to propylene oxide comprising:
a) a support of alkaline earth carbonate; and
b) a catalytically effective amount of silver
wherein the alkaline earth carbonate is of the general formula ACO₃ where A is beryllium, calcium, strontium, magnesium, barium or radium and is scalenohedral, irregular rhombohedral, acicular or prismatic.

2. The catalyst of claim 1 wherein the alkaline earth carbonate is calcium carbonate.

3. The catalyst of claim 1 wherein the surface area of the alkaline earth carbonate is from about 1 m²/g to about 30 m²/g.

4. The catalyst of claim 1 wherein the alkaline earth carbonate contains elements chosen from the group consisting of iron, magnesium, strontium, barium, lead, sodium, potassium and sulfur present up to 5% by weight.

5. The catalyst of claim 1 wherein the support additionally contains an alkaline earth oxide of the general formula BO where B is beryllium, calcium, strontium, magnesium, barium or radium, A and B being the same or different.

6. The catalyst of claim 5 wherein the alkaline earth oxide is calcium oxide.

7. The catalyst of claim 1 wherein the silver is present in an amount from about 2 percent to 80 percent by weight.

8. The catalyst of claim 7 wherein the silver is present in an amount from about 10 percent to 70 percent by weight.

9. The catalyst of claim 7 wherein the silver is present in an amount from about 30 percent to 70 percent by weight.

10. The catalyst of claim 7 wherein the silver is present in an amount about 54 percent by weight.

11. The catalyst of claim 1 additionally comprising an alkali metal or a halogen.

12. The catalyst of claim 11 wherein the alkali metal is chosen from the group consisting of potassium, sodium, rubidium and cesium.

13. The catalyst of claim 11 wherein the halide is chlorine.

14. The catalyst of claim 11 wherein the alkali metal or the halogen is present in an amount from 0.1 to 5% by weight.

15. The catalyst of claim 14 wherein the alkali metal is present in the amount of from about 1 to 5% by weight.

16. The catalyst of claim 15 wherein the alkali metal is present in the amount of from about 3% by weight.

17. The catalyst of claim 14 wherein the halide is present in the amount from about 0.01 to 1.0% by weight.

18. The catalyst of claim 17 wherein the halide is present in the amount from about 0.05 to 0.5% by weight.

19. The catalyst of claim 1 additionally comprising a metal chosen from the group consisting of gold, tungsten, rhenium, molybdenum, thallium, yttrium, barium, cerium, cobalt, indium and niobium.

20. The catalyst of claim 19 wherein the metals are present in the amount from about 0.1 to 5.0% by weight

21. The catalyst of claim 17 wherein the metals are present in the amount from about 0.1 to 2% by weight.

22. A process for making a catalyst for oxidation of propylene to propylene oxide comprising:
a) contacting an alkaline earth carbonate with a solution, slurry, paste or gel containing a silver compound;
b) removing liquid from the solution, slurry, paste or gel to form a residue of solid particles;
c) drying the solid particles; and
d) calcining the solid particles.

23. The process of claim 22 wherein the silver compound is an oxide, a salt or carboxylate.

24. The process of claim 22 wherein the silver compound is silver oxide, silver nitrate, silver carbonate, silver acetate, silver propionate, silver butyrate, silver oxalate, silver malonate, silver malate, silver, maleate, silver lactate, silver citrate or silver phthalate.

25. The process of claim 22 additionally comprising adding alkali metal salts in step a), after step c) or after step d).

26. The process of claim 25 wherein the alkali metals are chosen from the group consisting of potassium, sodium, rubidium and cesium.

27. The process of claim 25 wherein the alkali metal salts are carbonates, nitrates or nitrites.

28. The process of claim 25 wherein the alkali metal salt is potassium nitrate.

29. The process of claim 22 additionally comprising adding a halide compound in step a), after step c) or after step d).

30. The process of claim 29 wherein the halide compound is silver chloride.

31. The process of claim 22 additionally comprising an oxide, acid, carbonate, sulfate, halide, oxyhalide, hydroxyhalide, hydroxide and sulfide of gold, tungsten, rhenium, molybdenum, fluorine, thallium, yttrium, barium, cerium, cobalt, indium or niobium in step a), after step c) or after step d).

32. The process of claim 22 additionally comprising adding an acid after step a).

33. The process of claim 29 wherein the acid is chosen from the group consisting of oxalic acid, propionic acid, malonic acid, citric acid, glycolic acid or mixtures thereof.

34. The process of claim 22 wherein the liquid is removed by filtration, evaporation or spray drying.

35. The process of claim 22 wherein drying is in air or an inert gas.

36. The process of claim 35 wherein drying is for one hour to twenty-four hours at a temperature from 110° C. to 250° C.

37. The process of claim 36 wherein drying is for four hours at 250° C.

38. The process of claim 22 wherein calcining is at a temperature of from about 100° C. to about 500° C. for a time of from about one hour to about four hours.

39. The process of claim 38 wherein the temperature is about 250° C. for six hours.

40. The process of claim 39 wherein the temperature is 110° C. for one hour and then increased by 5° C./min to a temperature of 300° C. for additional calcination for four hours.

41. The process of claim 22 additionally comprising a reducing agent in step d).

42. The process of claim 41 wherein the reducing agent is hydrogen.

43. A process for producing propylene oxide from propylene and oxygen comprising contacting a catalyst comprising:
a) a support of alkaline earth carbonate; and
b) a catalytically effective amount of silver
wherein the alkaline earth carbonate is of the general formula ACO₃ where A is calcium, strontium, magnesium or barium and is scalenohedral, irregular rhombohedral, acicular or prismatic;
with propylene and oxygen under reaction condition to selectively convert propylene to propylene oxide.

44. The process of claim 43 wherein the reaction conditions comprise temperatures from about 180° C. to 350° C.

45. The process of claim 44 wherein the temperature is from 200° C. to 300° C.

46. The process of claim 43 wherein the reaction conditions comprise a pressure from about 1 atmosphere to 30 atmospheres.

47. The process of claim 46 wherein the pressure is from 1 atmosphere to 5 atmospheres.

48. The process of claim 46 wherein the pressure is from 10 atmosphere to 20 atmospheres.

49. The process of claim 43 wherein propylene is present in the amount from about 2 to about 50% by volume

50. The process of claim 49 wherein propylene is present in the amount from about 10 to about 30%

51. The process of claim 50 wherein propylene is present in the amount from about 10% to about 20% by volume.

52. The process of claim 43 wherein oxygen is present in amount from about 2 to about 50% by volume.

53. The process of claim 52 wherein oxygen is present in the amount from about 10 to 25%.

54. The process of claim 53 wherein oxygen is present in the amount from about 15% by volume.

55. The process of claim 43 additionally comprising carbon dioxide.

56. The process of claim 55 wherein the carbon dioxide is present in the amount from about 1 to about 50% by volume.

57. The process of claim 56 wherein the carbon dioxide is present in the amount about 5 to about 50% by volume.

58. The process of claim 57 wherein the carbon dioxide is present in the amount of about 10% by volume.

59. The process of claim 43 additionally comprising a gaseous nitrogen oxide species.

60. The process of claim 59 wherein the gaseous nitrogen oxide species is nitrogen dioxide (NO₂), nitric oxide (NO), nitrogen peroxide (N₂O₄) or nitrogen trioxide (N₂O₃).

61. The process of claim 60 wherein the gaseous nitrogen oxide species is nitric oxide.

62. The process of claim 59 wherein the gaseous nitrogen oxide species is present in the amount of from 1 to 2000 ppm.

63. The process of claim 62 wherein the gaseous nitrogen oxide species is present in the amount of 20 to 500 ppm.

64. The process of claim 63 wherein the gaseous nitrogen oxide species is present in the amount of about 50 ppm.

65. The process of claim 43 additionally comprising an organic halide.

66. The process of claim 65 wherein the organic halide is alkyl halides.

67. The process of claim 66 wherein the alkyl halide is ethylene dichloride, ethyl chloride, vinyl chloride, methyl chloride and methylene chloride.

68. The process of claim 67 wherein the alkyl halide is ethyl chloride.

69. The process of claim 66 wherein the organic halide is present in the amount of from about 1 to 2000 ppm.

70. The process of claim 69 wherein the organic halide is present in the amount of from about 20 to 500 ppm.

71. The process of claim 70 wherein the organic halide is present in the amount of from about 50 to 500 ppm.

72. The process of claim 43 wherein the alkaline earth carbonate is calcium carbonate.

73. The process of claim 43 wherein the alkaline earth carbonate contains elements chosen from the group consisting of iron, magnesium, strontium, barium, lead, sodium, potassium and sulfur present up to 5% by weight.

74. The process of claim 43 wherein the support additionally contains an alkaline earth oxide of the general formula BO where B is beryllium, calcium, strontium, magnesium, barium or radium, A and B being the same or different.

75. The process of claim 74 wherein the alkaline earth oxide is calcium oxide.

76. The process of claim 43 wherein the silver is present in an amount from about 2 percent to 80 percent by weight.

77. The process of claim 76 wherein the silver is present in an amount from about 10 percent to 70 percent by weight.

78. The process of claim 77 wherein the silver is present in an amount from about 30 percent to 70 percent by weight.

79. The process of claim 78 wherein the silver is present in an amount of about 54 percent by weight.

80. The process of claim 43 wherein the catalyst additionally comprises an alkali metal or a halogen.

81. The process of claim 80 wherein the alkali metal is potassium.

82. The process of claim 80 wherein the halide is chlorine.

83. The process of claim 80 wherein the alkali metal or the halogen is present in an amount from 0.1 to 5% by weight.

84. The process of claim 43 wherein the catalyst additionally comprises a metal chosen from the group consisting of gold, tungsten, rhenium, molybdenum, fluorine, thallium, yttrium, barium, cerium, cobalt, indium and niobium.

85. The process of claim 17 wherein the metals are present in the amount from about 0.1 to 5.0% by weight.
